(19) United States
(12) Patent Application Publication
(10) Pub. No.: US 2026/0048231 A1
(43) Pub. Date: Feb. 19, 2026

(54) CATHETER SHAFT AND METHODS OF MAKING A CATHETER SHAFT

(71) Applicant: Boston Scientific Scimed, Inc., Maple Grove, MN (US)

(72) Inventors: Emily Rose Whitwam, Blaine, MN (US); Jason John Matteson, JR., Beldenville, WI (US); Kevin Martin, New Brighton, MN (US); Matthew Nelson Frost, New Brighton, MN (US)

(21) Appl. No.: 19/300,499

(22) Filed: Aug. 14, 2025

Related U.S. Application Data
(60) Provisional application No. 63/684,080, filed on Aug. 16, 2024.

(51) Int. Cl. A61M 25/00

(52) U.S. Cl. CPC A61M 25/005; A61M 25/0012

(57) ABSTRACT

A medical catheter and its manufacturing method are presented. The catheter features a shaft with a continuous reinforcing braid and a polymeric jacket. Exposed braid grooves are created by selectively removing portions of the polymeric jacket, enabling a mechanical connection of structural elements directly to the exposed braid. These structural elements, such as a reinforcing assembly or an articulation structure, are securely connected through the mechanical connection at the attachment location. This integration enhances the catheter's stability, maneuverability by establishing a direct, seamless connection between the structural element and the catheter shaft creating a catheter with improved performance. The disclosed invention advances catheter design by providing efficient means of integrating structural elements within the catheter shaft.

### CROSS REFERENCE TO RELATED APPLICATIONS

The present application claims priority to U.S. Provisional Patent Application Ser. No. 63/684,080, filed Aug. 16, 2024, the disclosure of which is incorporated herein in its entirety.

### TECHNICAL FIELD

The present invention relates generally to medical catheters and methods for manufacturing medical catheters. More specifically, to medical catheters with enhanced structural integration and performance through mechanical connections of structural elements such as a reinforcing assembly or an articulation structure.

### BACKGROUND

Medical catheters rely on the integration of various structural elements, such as a reinforcing assembly for steering wire guidance or an articulation structure for controlled deflection, to achieve optimal maneuverability and performance. However, securely anchoring these components within the catheter shaft can be challenging. Conventional methods of integrating structural elements often involve bonding them to the inner surface of the catheter shaft using adhesives or thermal techniques. However, these approaches may not provide sufficient mechanical stability or durability, especially in regions of the catheter subjected to high stress during steering and navigation. In addition, the use of adhesives or thermal bonding can create variability in the outer diameter of the catheter shaft, as well as localized stiffness or weakness in the catheter shaft, disrupting the desired gradual transition of mechanical properties along its length. These inefficiencies can impede positioning and performance capabilities of the catheter.

Therefore, there is a need for improved devices and methods for securely integrating structural elements within a medical catheter while maintaining a smooth, continuous profile and optimizing mechanical performance. The present invention addresses these needs by utilizing exposed portions of the catheter shaft's reinforcing braid as anchoring points for structural elements. By selectively exposing the underlying reinforcing braid through a removal process and mechanically connecting structural elements directly to the braid, allows for structural integration without compromising the catheter's flexibility or introducing abrupt changes in stiffness. This approach enhances the structural continuity between the catheter shaft and structural elements such as the reinforcing assembly or articulation structure, resulting in improved steering responsiveness and overall catheter performance.

### SUMMARY

In Example 1, a medical catheter comprising a handle configured for manipulation by a user, and a flexible shaft comprising a reinforcing braid and a polymeric jacket disposed over the reinforcing braid, the flexible shaft having a proximal portion, and a distal portion, the shaft further comprising a structural element is positioned within the shaft. The structural element is mechanically connected to the reinforcing braid at an attachment location via a mechanical connection establishing a secure bond between the reinforcing braid and the structural element, and the mechanical connection is formed by exposing the reinforcing braid by selective removal of the polymeric jacket material, and mechanically connecting the braid to the structural element.

In Example 2, the medical catheter of Example 1, wherein the structural element is configured to provide reinforcement to the flexible shaft or transmit forces along the flexible shaft.

In Example 3, the medical catheter of Example 1, wherein the mechanical connection comprises a welded connection between the reinforcing braid and the structural element.

In Example 4, the medical catheter of Example 1, wherein the selective removal of the polymeric jacket is performed at any location along the length of the flexible shaft.

In Example 5, the medical catheter of Example 1, wherein the polymer jacket is removed to expose the reinforcing braid at one or more locations along the flexible shaft, allowing for the mechanical connection of the structural element at each of the one or more locations.

In Example 6, the medical catheter of Example 1, wherein the selective removal of the polymeric jacket is controlled to create a desired size and shape of exposure of the reinforcing braid.

In Example 7, the medical catheter of Example 1, wherein the structural element is a reinforcing assembly, wherein the reinforcing assembly is positioned within the proximal portion of the shaft, wherein the reinforcing assembly comprises a pair of diametrically opposed helical reinforcing sleeves extending through the proximal portion of the shaft, and an anchor ring attached to a distal end of the reinforcing sleeves, the anchor ring comprising a ring structure.

In Example 8, the medical catheter of Example 7, wherein the reinforcing sleeves establish reinforced passageways configured to provide radial and lateral support for one or more steering elements extending from the proximal region to the distal region.

In Example 9, the medical catheter of Example 7, wherein the anchor ring includes passageways for the one or more steering elements extending from the proximal region to the distal region.

In Example 10, the medical catheter of Example 9, wherein the anchor ring of the reinforcing assembly is positioned in close proximity to the attachment location.

In Example 11, the medical catheter of Example 10, wherein the anchor ring is connected through the mechanical connection to the reinforcing braid at the attachment location.

In Example 12, the medical catheter of Example 1, wherein the structural element is an articulation structure positioned within the distal portion of the shaft, the articulation structure having a proximal end, wherein the articulation structure comprises a plurality of longitudinally-arranged articulation elements, one or more steering wire lumens, wherein the steering wire lumens extend through the articulation structure, and one or more reinforcing members extending through the articulation elements.

In Example 13, the medical catheter of Example 1, wherein the structural element is a single helical cable that comprises a proximal end and distal end, wherein the single helical cable extends longitudinally through the proximal shaft, and wherein the single helical cable provides structural support to the proximal shaft.

In Example 14, the medical catheter of Example 13, wherein the distal end of the single helical cable is positioned in close proximity to the attachment location.

In Example 15, the medical catheter of Example 14, wherein the distal end of the single helical cable is connected through the mechanical connection to the reinforcing braid at the attachment location.

In Example 16, a medical catheter comprising a handle configured for manipulation by a user, and a flexible shaft. The flexible shaft comprises a reinforcing braid and a polymeric jacket disposed over the reinforcing braid, the flexible shaft having a proximal portion, and a distal portion, the shaft further comprising a reinforcing assembly extending within the proximal portion of the shaft, wherein the reinforcing assembly comprises an anchor ring and a pair of diametrically opposed reinforcing sleeves attached to and extending proximally from the anchor ring within the proximal portion of the shaft. The anchor ring is mechanically connected to an attachment location via a mechanical connection establishing a secure bond between the shaft and the anchor ring, and the mechanical connection is formed by exposing the reinforcing braid by selectively removing the polymeric jacket material and mechanically connecting the braid to the anchor ring.

In Example 17, the medical catheter of Example 16, wherein the mechanical connection is a welded connection between the reinforcing braid and the anchor ring.

In Example 18, the medical catheter of Example 16, further comprising a pair of diametrically opposed steering elements each extending through a respective one of the reinforcing sleeves, wherein the reinforcing sleeves each provide radial and lateral support for the steering elements extending therethrough.

In Example 19, the medical catheter of Example 18, wherein the anchor ring includes a pair of diametrically opposed passageways aligned with the passageways of the reinforcing sleeves, and when each of the steering elements extends through a respective one of the passageways in the anchor ring.

In Example 20, the medical catheter of Example 19, wherein the anchor ring of the reinforcing assembly is positioned in close proximity to the attachment location.

In Example 21, the medical catheter of Example 20, wherein the shaft further comprises an articulation structure positioned within the distal portion of the shaft, the articulation structure having a proximal end positioned distally adjacent to the anchor ring.

In Example 22, a medical catheter shaft comprising a proximal portion, a distal portion, a reinforcing braid extending along the proximal and distal portions, a polymeric jacket disposed over the reinforcing braid, and a structural element positioned within the shaft, wherein the structural element is mechanically connected to the reinforcing braid at an attachment location via a mechanical connection establishing a secure bond between the reinforcing braid and the structural element, and wherein the mechanical connection is formed by exposing the reinforcing braid by selective removal of the polymeric jacket material and mechanically connecting the braid to the structural element.

In Example 23, the medical catheter shaft of Example 22, wherein the mechanical connection comprises a welded connection between the reinforcing braid and the structural element.

In Example 24, the medical catheter shaft of Example 23, wherein the selective removal of the polymeric jacket is controlled to create a desired size and shape of exposure of the reinforcing braid.

In Example 25, the medical catheter shaft of Example 23, wherein the structural element is a reinforcing assembly, wherein the reinforcing assembly is positioned within the proximal portion of the shaft, wherein the reinforcing assembly comprises a pair of diametrically opposed helical reinforcing sleeves extending through the proximal portion of the shaft, and an anchor ring attached to a distal end of the reinforcing sleeves, the anchor ring comprising a ring structure.

In Example 26, the medical catheter shaft of Example 25, wherein the mechanical connection connects the reinforcing braid and the anchor ring.

In Example 27, the medical catheter shaft of Example 26, wherein the reinforcing sleeves establish reinforced passageways configured to provide radial and lateral support for one or more steering elements extending from the proximal region to the distal region.

In Example 28, the medical catheter shaft of Example 27, wherein the anchor ring includes passageways for the one or more steering elements extending from the proximal region to the distal region.

In Example 29, the medical catheter shaft of Example 23, wherein the structural element is an articulation structure positioned within the distal portion of the shaft, the articulation structure having a proximal end, wherein the articulation structure comprises a plurality of longitudinally-arranged articulation elements, one or more steering wire lumens, wherein the steering wire lumens extend through the articulation structure, and one or more reinforcing members extending through the articulation elements.

In Example 30, the medical catheter shaft of Example 28, wherein the mechanical connection connects the reinforcing braid to the proximal end of the articulation structure.

In Example 31, the medical catheter shaft of Example 23, wherein the structural element is a single helical cable that comprises a proximal end and distal end, wherein the single helical cable extends longitudinally through the proximal shaft, and wherein the single helical cable provides structural support to the proximal shaft, and wherein the mechanical connection connects the reinforcing braid to the distal end of the single helical cable.

In Example 32, a method of producing a shaft for a medical catheter, the method comprising providing a flexible tubular member comprising a reinforcing braid and a polymeric jacket disposed over the reinforcing braid, the flexible tubular member having a proximal portion, and a distal portion, removing a portion of the polymeric jacket at an attachment location to expose a portion of the reinforcing braid, positioning a structural element within the flexible tubular member adjacent to the exposed portion of the reinforcing braid, and connecting the structural element to the exposed reinforcing braid at the attachment location to establish a secure bond between the structural element and the flexible tubular member.

In Example 33, the method of Example 32, wherein connecting the structural element comprises welding the structural element to the reinforcing braid.

In Example 34, the method of Example 33, wherein the structural element comprises a reinforcing assembly comprising a pair of diametrically opposed helical reinforcing sleeves and an anchor ring attached to a distal end of the reinforcing sleeves, and wherein positioning the structural element includes positioning the anchor ring adjacent to the attachment location, and wherein connecting the structural element includes welding the anchor ring to the reinforcing braid.

In Example 35, the method of Example 33, wherein the structural element comprises an articulation structure having a proximal end, and wherein positioning the structural element includes positioning the proximal end of the articulation structure adjacent to the attachment location, and wherein connecting the structural element includes welding the proximal end of the articulation structure to the reinforcing braid.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is an illustration of an example medical catheter, consistent with various aspects of the present disclosure.

FIG. 2A is an illustration of the medical catheter, shown in FIG. 1, as deflected in a first direction, consistent with various aspects of the present disclosure.

FIG. 2B is an illustration of the medical catheter, shown in FIG. 1, as deflected in a second direction, consistent with various aspects of the present disclosure.

FIG. 3 is a detailed illustration of a portion of a side-view illustration of the medical catheter, consistent with various aspects of the present disclosure.

FIG. 4 is an illustration of a reinforcing assembly enclosed in the medical catheter with a anchor ring at an attachment location, consistent with various aspects of the present disclosure.

FIG. 5A is a top-view illustration of a reinforcing assembly of the medical catheter, consistent with various aspects of the present disclosure.

FIG. 5B is a partial isometric illustration of an alternative reinforcing assembly of the medical catheter, consistent with various aspects of the present disclosure.

FIG. 6A is an illustration of an articulation structure positioned at an attachment location and reinforcing assembly with a anchor ring positioned at another attachment location enclosed in the medical catheter, consistent with various aspects of the present disclosure.

FIG. 6B is an illustration of an articulation structure positioned at an attachment location and a single helical cable positioned at another attachment location enclosed in the medical catheter, consistent with various aspects of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

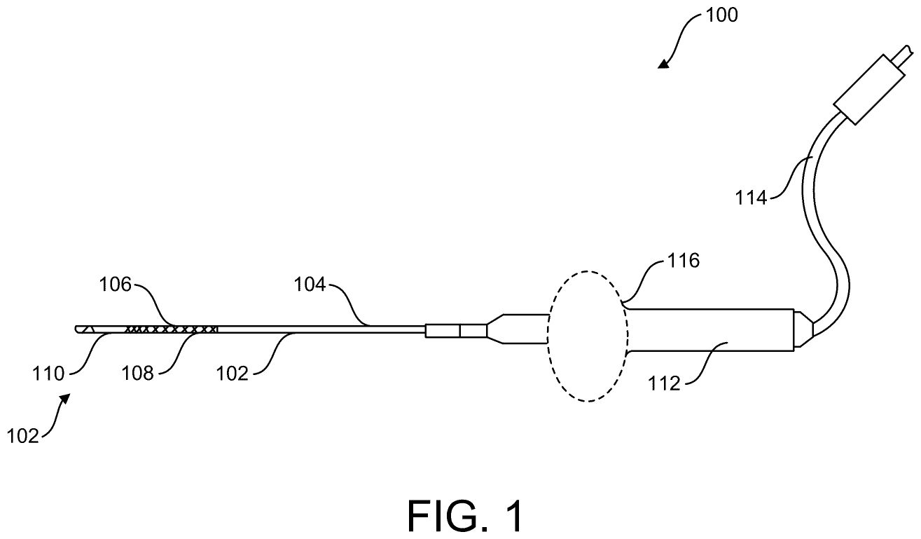

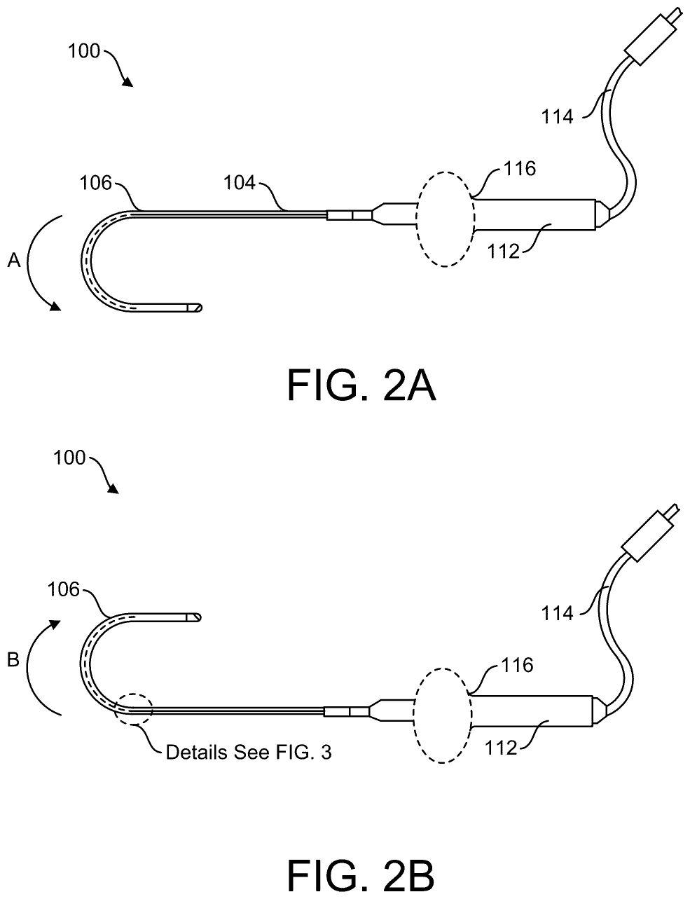

FIG. 1, FIGS. 2A and 2B are illustrations of an example medical catheter 100, consistent with various aspects of the present disclosure. As shown in FIG. 1, the medical catheter 100 may be a steerable medical catheter 100. In certain instances, the medical catheter 100 is steerable in one direction (e.g., direction A as shown in FIG. 2A) or in another direction (e.g., direction B as shown in FIG. 2B). The medical catheter 100 generally includes a tubular shaft 102 having a proximal portion 104 and a distal portion 106 that is sized and configured for placement and manipulation within in a target area of a patient. The distal portion 106 may be steerable. As shown, the distal portion 106 further includes a deflection region 108 and a distal end 110.

In certain instances, the tubular shaft 102 extends from a distal portion of a handle 112. An electrical cable or other suitable connector 114 extending from a proximal end of the handle 112 may be coupled to a source of energy or other equipment (not shown in FIG. 1) for transmitting one or more ablation signals. A steering actuator 116, such as a rotatable knob or plunger that may be arranged at the handle 112, may be manipulated by a physician to deflect or position the steerable distal portion 106 of the tubular shaft 102.

As shown in FIGS. 2A and 2B, the medical catheter 100 is of the deflectable or steerable type, such that during use, the deflection region 108 can be deflected or curved by a user to facilitate locating the distal end 110 at a desired target location within the heart. In embodiments, deflection of the deflection region 108 can be accomplished by manipulation of the steering actuator 118, which is operatively connected to steering elements (e.g., wires, tendons, ribbons, and the like) extending within and attached (directly or indirectly) to the shaft 102 at a location within the distal portion 106. The particular mode and structure for deflecting the deflection region 108 is not critical to the present disclosure, and so any technique, whether now known or later developed, can be employed within the scope of the present disclosure.

In use, deflecting or curving the distal portion 106 may impart a torsional force that could torque or twist the distal portion 106 away from or out of the target location. The deflection region 108 may torque out of plane from the plane in which the distal portion 106 was arranged prior to deflection due to the tension on the curvature of the medical catheter 100 within vasculature.

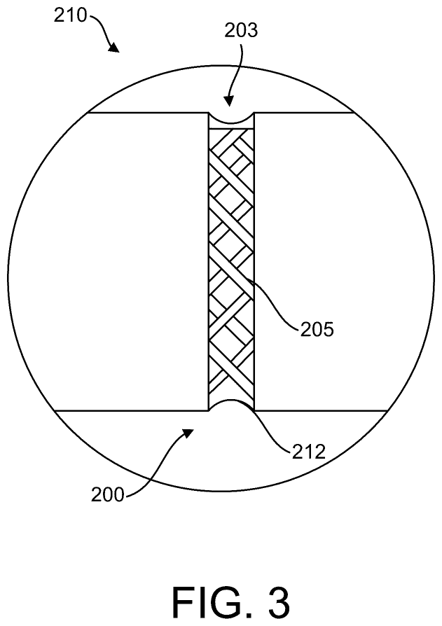

FIG. 3 is an enlarged detail view of the medical catheter 100 illustrating an attachment location 200 on the catheter shaft 102. The catheter shaft 102 is composed of a reinforcing braid 205 and a polymeric jacket 210 disposed over the reinforcing braid 205, wherein the reinforcing braid 205 is formed of a plurality of interwoven wires that are woven, knitted, entwined or otherwise interlaced together. The skilled artisan will recognize that the use of jackets in catheter construction is well known, and the particular details of the braid/jacket construction are not of critical importance to the present disclosure. In embodiments, the aforementioned braid can be omitted, or alternatively, other constructions, e.g., reinforcing coils, can be employed to enhance the structural and torsional strength of the jacket.

As shown, the selective removal of the polymeric jacket at any location along the length of the shaft to expose the underlying reinforcing braid 205, allows a user flexibility to connect a structural element that is positioned within the shaft through the exposed braid at the attachment location 200. This removal process may be done by laser ablation wherein a user may strategically remove the polymeric jacket 210 around the circumference of the shaft 102 to expose the underlying reinforcing braid 205. The length, width, and depth of the exposed portion of the reinforcing braid 205 may be tailored to a desired size to accommodate the integration of different structural elements. In some instances, the removal of the polymeric jacket 210 may create an exposed braid groove 212 at the attachment location 200 of the shaft 102. The selective removal of the polymeric jacket 210 may also create a braided gap 203 between the remaining portions of the polymeric jacket 210 wherein the length of the braided gap 203 may be controlled by the removal process. In various embodiments, the braided gap 203 may have a length of about 0.020 inches to 0.015 inches.

The selective removal of polymeric material of the polymeric jacket 210 exposes the underlying reinforcing braid 205 providing access to allow the integration of structural elements by a mechanical connection to the reinforcing braid 205 at the attachment location 200. The internal structural elements may comprise a reinforcing assembly, an articulation structure, or of similar structural elements. The mechanical connection to integrate the structural element to the reinforcing braid 205 may be done through a welding process, soldering process, adhesive process, polymer reflow process, or other processes that may enhance the connection between the structural element and the reinforcing braid 205 at the attachment location. In some instances, the existing polymeric jacket 210 may be reflowed over the exposed braid groove 212 and the braid gap 203 to seal and encapsulated the attachment location 200. The reflowed polymeric material from the existing polymeric jacket 210 forms a smooth, continuous surface over the exposed reinforcing braid 204 and the integrated structural elements.

In some cases, if the braided gap 203 is too wide, the existing polymeric jacket 210 may not provide sufficient material to completely cover the exposed reinforcing braid 205 when reflowed. In such instances, additional polymeric material may need to be added to the reflowed polymeric jacket 210 to ensure complete coverage and sealing of the attachment location 200. The additional polymeric material may be of the same composition as the existing polymeric jacket 210 or a compatible material.

In some embodiments, a user may strategically remove the polymeric jacket 210 partially or fully by laser ablation or of similar removal process such that the length, width, and depth of the exposed portion of the reinforcing braid 205 may be tailored to a desired size to accommodate integration of different structural elements to the shaft.

In some embodiments, the removal process may involve mechanical cutting, chemical etching, thermal ablation, and other suitable removal processes.

In some embodiments, creation of one or more exposed portion of the reinforcing braid 205 along the catheter shaft 102 may be formed to allow a user access to mechanically connect the structural element or various structural elements at various attachment locations.

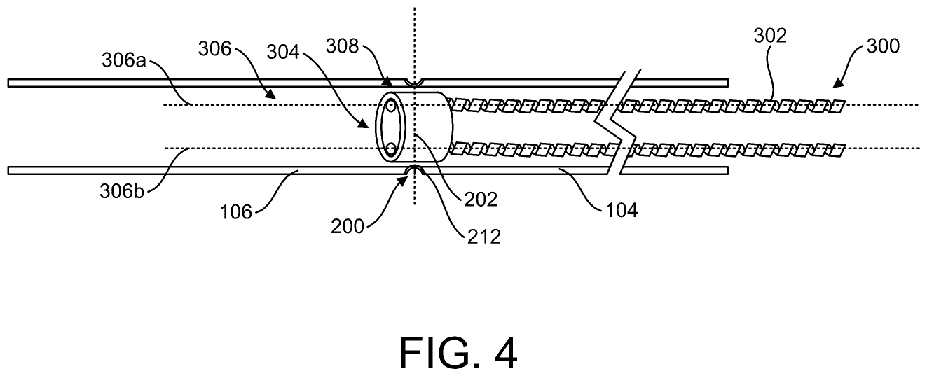

Referring now to FIG. 4, a reinforcing assembly 300 is introduced within the medical catheter 100, wherein the selective removal of portions of the polymeric jacket 210 and mechanical connection 202 of the reinforcing assembly 300 is performed at the attachment location 200. The reinforcing assembly 300, is a structural element within the medical catheter that facilitates steering control and maneuverability with the integration of reinforcing sleeves 302 which may act as a reinforced steering wire guidance system within the catheter shaft, allowing for controlled deflection of the distal portion 106 while providing support and stability to the proximal portion 104. The reinforcing assembly 300 is positioned within the proximal portion 104 of the medical catheter shaft 102 and extends into the distal portion 106 and comprises reinforcing sleeves 302 and a anchor ring 304, wherein the anchor ring 304 may be composed from a metallic material or of a similar alloy. The reinforcing sleeves 302 may comprise a pair of diametrically opposed helical coils that extends longitudinally through the proximal portion 104 of the shaft 102. Further, the reinforcing sleeves 302 create dedicated passageways for one or more steering elements (e.g., wires, tendons, ribbons, and the like) 306, wherein the one or more steering element may comprise a first steering element 306a and a second steering element 306b. The one or more steering elements 306 are located in diametrically opposite positions and are operatively connected to the steering actuator 118 (FIG. 1) to facilitate the deflection of at least the deflection region 108 of the shaft 102 as known in the conventional manner. Additionally, the reinforcing sleeves 302 may provide radial and lateral support and guidance for the one or more steering elements 306 by encasing them within the helical coils. The protective structure may help minimize the impact of any compressive or any other forces that may occur during catheter manipulation allowing the steering elements to maintain their integrity and functionally for consistent and precise deflection of the deflection region 108.

Further, at a distal end of the reinforcing assembly 308, the reinforcing sleeves 302 are securely attached to the anchor ring 304, wherein the anchor ring 304 includes passageways for the one or more steering elements 306 extending from the proximal region to the distal region. The anchor ring is shown with a smooth, non-grooved anchor ring 304, wherein the anchor ring may be positioned underneath and at center of, or in close proximity to the attachment location 200 of the catheter shaft 102 serving as an anchoring point. The anchor ring 304 may be connected to the reinforcing braid 205 at the attachment location 200 by the mechanical connection 202. The mechanical connection may be done through a welding process, soldering process, adhesive process, polymer reflow process, or other processes that may allow and enhance the connection between the anchor ring 304 and the exposed underlying reinforcing braid 205. In some instances, the existing polymeric jacket 210 may be reflowed over the exposed braid groove 212 and the braid gap 203 to seal and encapsulate the attachment location 200. The secure attachment of the anchor ring to the exposed reinforcing braid allows for a seamless transfer of steering forces from the proximal portion 104 to the distal portion 106, allowing control and maneuverability of the medical catheter 100.

In an example embodiment, the anchor ring may be illustrated with a grooved anchor ring. The grooved anchor ring may feature a circumferential groove, wherein the circumferential groove may accommodate an adhesive. The circumferential groove allows an adhesive to fill the space, creating a connection between the anchor ring and the reinforcing braid 205 at the attachment location 200. The adhesive may flow through the braided gap 203 and flow into the groove, filling the voids and forming a strong bond that prevents slippage and maintains the integrity of the reinforcing assembly 300. Further, in some instances, the existing polymeric jacket 210 may be reflowed over the braid gap 203 and exposed braid groove 212 to seal and encapsulate the attachment location 200. The choice between a grooved anchor ring and the smooth anchor ring 304 (FIG. 4) may depend on various factors, including the desired level of attachment strength, manufacturing feasibility, and specific requirements of the medical catheter application.

The strategic positioning and mechanical connection of the anchor ring 304 to the reinforcing braid 205 at the attachment location 200 allows transfer of steering forces from the proximal portion 104 to the distal portion 106. The mechanical connection may help with overall control and maneuverability of the medical catheter 100 while maintaining a smooth transition between the proximal portion and distal portion without compromising the catheter's flexibility or introducing abrupt changes in stiffness enabling precise navigation through challenging anatomical structures.

In some embodiments, the reinforcing assembly 300 may be adapted to suit specific catheter designs and applications. Variations in the number, configuration, and/or material composition of the reinforcing sleeves 302 and anchor ring 304 may be employed to optimize performance characteristics. For example, the helical coils of the reinforcing sleeves may be designed with different diameters and materials, or the anchor ring may be constructed of various materials to achieve the desired strength, and flexibility.

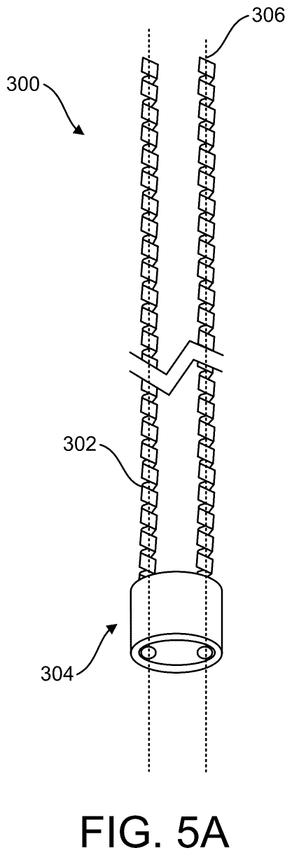

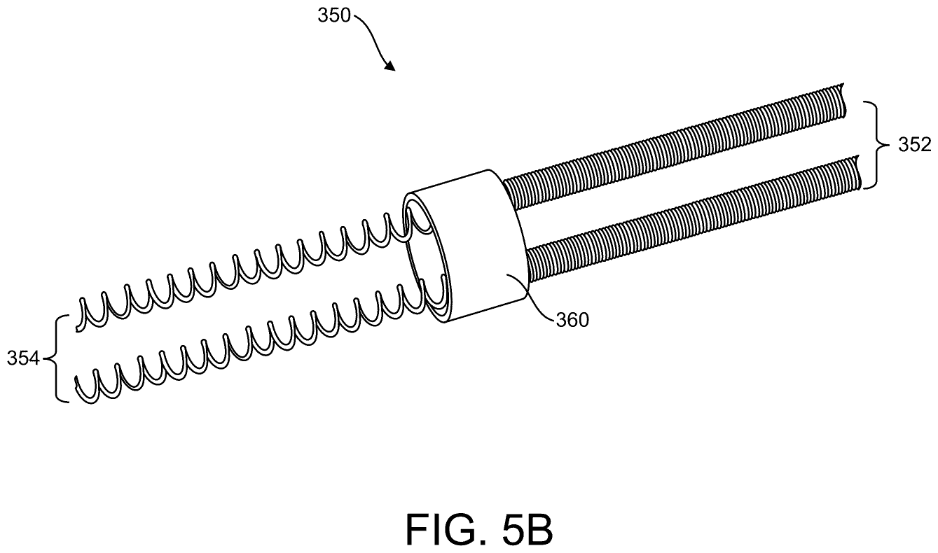

FIG. 5A is a top view illustration of the reinforcing assembly 300 as previously described in FIG. 4. As shown, the reinforcing assembly includes the reinforcing sleeves 302 wherein the reinforcing sleeves 302 create dedicated passageways 320, 322 for the one or more steering elements 306. The one or more steering elements 306 may be located in diametrically opposite positions and are operatively connected to the steering actuator 118 (FIG. 1) to facilitate the deflection of at least the deflection region 108 of the tubular shaft 102 as known in the conventional manner.

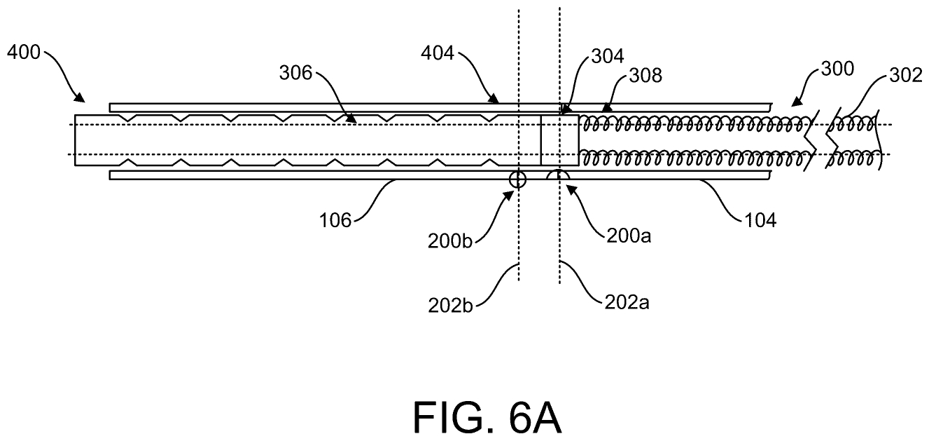

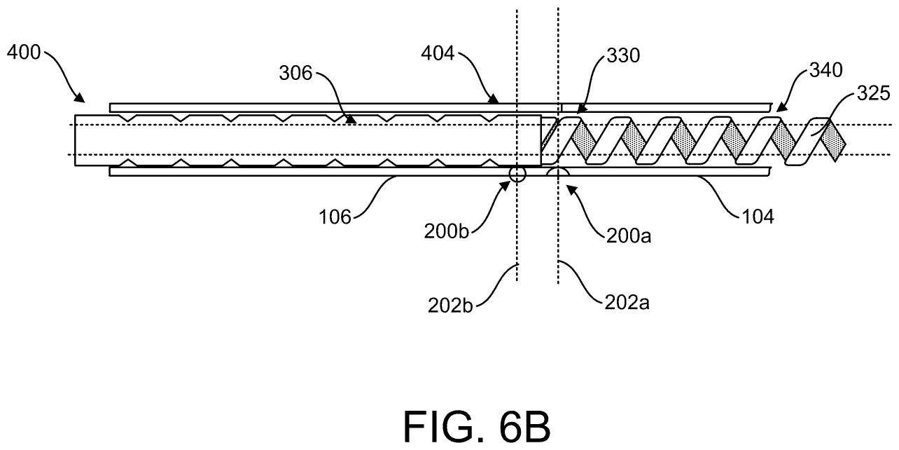

FIG. 5B is a partial isometric illustration of an alternative reinforcing assembly 350, according to various embodiments. As shown, the reinforcing assembly 350 includes a pair of coiled proximal reinforcing sleeves 352, a pair of coiled distal reinforcing sleeves 354, and a anchor ring 360 disposed therebetween. In the illustrated embodiment, the proximal reinforcing sleeves 352 have a relatively tight, or even close, pitch, and can correspond functionally and structurally to the reinforcing sleeves 302 described above. Additionally, the anchor ring 360 corresponds to the anchor ring 202 described above. As further shown, the distal pair of reinforcing sleeves 354 are attached at their proximal ends to and extend distally from the anchor ring 360. Each of the reinforcing sleeves 352 is axially aligned with one of the reinforcing sleeves 354 across the anchor ring 360 so as to form a continuous axial passage for slidably receiving a steering element as described above. In embodiments, an additional anchor ring may be attached to the distal ends of the pair of distal reinforcing sleeves 354 to maintain their special relationship and to facilitate attachment to the catheter shaft. In other embodiments, the distal reinforcing sleeves may be incorporated into an articulation element (e.g, the articulation structure 400 described below in conjunction with FIGS. 6A and 6B).

FIG. 6A and FIG. 6B introduces the creation of multiple attachment locations 200a and 200b along the catheter shaft 102 to allow for mechanical connection of various structural elements. This approach allows for the integration of structural elements such as the reinforcing assembly 300 and an articulation structure 400, which may contribute to the overall steering performance and stability of the catheter 100. The process may involve selectively removing portions of the polymeric jacket 210 at specific locations along the catheter shaft 102 to exposing reinforcing braids 205 at multiple attachment locations 200a and 200b, wherein the exposing of the reinforcing braids may create an exposed braid groove 212 and a braided gap 203.

As shown in FIG. 6A the reinforcing assembly 300 is positioned such that the anchor ring 304 sits underneath and at center of, or in close proximity to the first attachment location 200a. The anchor ring 304 may be connected by a first mechanical connection 202a to the reinforcing braid 205 at attachment location 200a securing the anchor ring 304 and the reinforcing assembly 300 to the catheter shaft 102. Similarly, the articulation structure 400 may be positioned within the distal portion of the shaft 106 a proximal end of the articulation structure 404 sits underneath and at center of, or in close proximity to the second attachment location 200b. The articulation structure 400 may be connected by a second mechanical connection 202b to the reinforcing braid 205 at attachment location 200b securing the articulation structure 400 to the catheter shaft 102. The mechanical connections 202a and 202b may be done through a welding process, soldering process, adhesive process, polymer reflow process, or other processes that may enhance the connection of the articulation structure and the reinforcing assembly to the reinforcing braids at the attachment locations 200a and 200b. In some instances, the existing polymeric jacket 210 may be reflowed over the exposed braid groove 212 and the braid gap 203 to seal and encapsulated the attachment locations 200a and 200b. The reflowed polymeric material from the existing polymeric jacket 210 forms a smooth, continuous surface over the exposed material without compromising the catheter's flexibility or introducing abrupt changes in stiffness.

FIG. 6B illustrates an alternative embodiment, wherein a single helical cable 340 is positioned within the proximal portion of the shaft 104. The single helical cable 340 extends longitudinally through the proximal portion 104 providing structural support or other functional and performance enhancements to the medical catheter 100. The distal end 330 of the single helical cable is positioned at center of, or in close proximity to the first attachment location 200a and connected to the exposed reinforcing braid 205 by the first mechanical connection 202a, securing the single helical cable 340 to the catheter shaft 102. The articulation structure 400 is positioned and connected to the second attachment location 202b in a similar manner as described in FIG. 6A. The proximal end of the articulation structure 404 sits underneath and at center of, or in close proximity to the second attachment location 200b. The articulation structure 400 may be connected by a second mechanical connection 202b to the reinforcing braid 205 at attachment location 200b securing the articulation structure 400 to the catheter shaft 102.

In some embodiments, the single helical cable 340 may have a ring structure attached its distal end 330. The ring structure may be similar to that of the anchor ring 304 described in FIG. 6A. The ring structure of the single helical cable may be positioned such that the ring sits underneath and at center of, or in close proximity to the first attachment location 200a. The ring of the single helical cable may be connected by a first mechanical connection 202a to the reinforcing braid 205 at attachment location 200a securing the ring structure and the single helical cable 340 to the catheter shaft 102. In some embodiments, the single helical cable 340 may be designed with different diameters and materials to achieve the desired strength, and flexibility in the catheter shaft.

In some embodiments, the proximal end of the articulation structure as shown in FIGS. 6A and 6B may include a metallic component or of similar alloy, such as a ring or collar, designed to support the articulation structure and allow for mechanical connection to the exposed braid at the attachment location. This metallic component may facilitate a smooth mechanical connection and provide additional strength and durability to the connection, allowing a stable integration of the articulation structure with the catheter shaft.

Further, the articulation structure 400 of FIGS. 6A and 6B facilitates predictable, highly planar deflection of the deflection region 108 by resisting torsional forces on the shaft 102 that would otherwise tend to cause the deflection region 108 to deflect. The articulation structure 400 may comprise series of interconnected plurality of longitudinally-arranged articulation elements that work together to achieve the desired deflection characteristics. These articulation elements may comprise a plurality of longitudinally arranged tubular segments, a plurality of connecting segments, plurality of articulation links, a plurality of joints, a plurality of reinforcing members, or combinations thereof. The articulation structure 400 may include one or more reinforcing members embedded within the articulation elements and extending through the articulation structure 400 wherein the reinforcing members may maintain a curved shape of the deflection region 108 during deflection and maintain repeatability of achieving the curved shape of the deflection region 108 during deflection. In an example embodiment the one or more reinforcing members may comprise a helically wound flat ribbon wire or stranded wire cables or of similar elements.

The articulation structure 400 may further comprise one or more steering wire lumens arranged in parallel to the passageways 320, 322 of the reinforcing assembly 300 in FIG. 6A and parallel to the single helical cable 325 in FIG. 6B, wherein the steering wire lumens may be configured to receive one or more steering elements 306 that are operatively connected to the steering actuator 118 (FIG. 1) to facilitate the deflection of at least the deflection region 108 of the tubular shaft 102 as known in the conventional manner. The one or more steering wire lumens may also be circumferentially offset from the one or more reinforcing members by about 90 degrees. The specific design and arrangement of these elements can vary depending on the desired deflection profile and the intended application of the medical catheter 100.

As shown, the articulation structure 400 may be positioned in close proximity to the reinforcing assembly 300 or the single helical cable 340. This placement allows transfer of steering forces from the reinforcing assembly 300 or the single helical cable 340 to the articulation structure 400, allowing smooth and controlled deflection of the distal portion. In response to a deflection force, the articulation structure 400 may bend in a direction A (FIG. 2A). In certain embodiments, the articulation structure 400 may bend in another direction B (FIG. 2B), opposite to that of direction A. Whether the articulation structure 400 is unidirectional or bidirectional is not of critical importance to the current invention.

The positioning of the attachment locations 200a and 200b, along with the placement of the reinforcing assembly 300, single helical cable 340, and articulation structure 400, can be tailored based on the specific design requirements of the catheter 100. Factors such as the desired deflection profile, the length of the distal portion 106, and the overall catheter stiffness can influence the optimal placement of these components. In some embodiments, additional attachment locations 200 may be created to accommodate mechanical connection to other structural elements or to provide multiple points for mechanical connection to a single structural element. As such, a user may strategically remove the polymeric material partially or fully by laser ablation or of similar removal process such that length, width, and depth of the exposed portion of the reinforcing braid 205 may be tailored to a desired size accommodate the integration of different structural elements to the shaft. Moreover, the pattern and arrangement of the exposed reinforcing braids 205 can be modified to achieve specific performance characteristics. For example, helical or staggered arrangements of the exposed reinforcing braids 205 could be utilized to optimize the flexibility and torsional rigidity of the catheter shaft 102 in specific region to allow for the secure integration of structural elements at multiple attachment locations while maintaining a smooth, continuous profile along the catheter shaft 102 contributing to the overall performance, maneuverability, and stability of the steerable medical catheter 100.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A medical catheter comprising:
a handle configured for manipulation by a user;
a flexible shaft comprising a reinforcing braid and a polymeric jacket disposed over the reinforcing braid, the flexible shaft having a proximal portion, and a distal portion, the shaft further comprising a reinforcing assembly extending within the proximal portion of the shaft, wherein the reinforcing assembly comprises an anchor ring and a pair of diametrically opposed reinforcing sleeves attached to and extending proximally from the anchor ring within the proximal portion of the shaft,
wherein the anchor ring is mechanically connected to an attachment location via a mechanical connection establishing a secure bond between the shaft and the anchor ring,
wherein the mechanical connection is formed by exposing the reinforcing braid by selectively removing the polymeric jacket material and mechanically connecting the braid to the anchor ring.

2. The medical catheter of claim 1, wherein the mechanical connection is a welded connection between the reinforcing braid and the anchor ring.

3. The medical catheter of claim 1, further comprising a pair of diametrically opposed steering elements each extending through a respective one of the reinforcing sleeves, wherein the reinforcing sleeves each provide radial and lateral support for the steering elements extending therethrough.

4. The medical catheter of claim 3, wherein the anchor ring includes a pair of diametrically opposed passageways aligned with the passageways of the reinforcing sleeves, and when each of the steering elements extends through a respective one of the passageways in the anchor ring.

5. The medical catheter of claim 4, wherein the anchor ring of the reinforcing assembly is positioned in close proximity to the attachment location.

6. The medical catheter of claim 5, wherein the shaft further comprises an articulation structure positioned within the distal portion of the shaft, the articulation structure having a proximal end positioned distally adjacent to the anchor ring.

7. A medical catheter shaft comprising:
a proximal portion;
a distal portion;
a reinforcing braid extending along the proximal and distal portions;
a polymeric jacket disposed over the reinforcing braid; and
a structural element positioned within the shaft,
wherein the structural element is mechanically connected to the reinforcing braid at an attachment location via a mechanical connection establishing a secure bond between the reinforcing braid and the structural element, and
wherein the mechanical connection is formed by exposing the reinforcing braid by selective removal of the polymeric jacket material and mechanically connecting the braid to the structural element.

8. The medical catheter shaft of claim 7, wherein the mechanical connection comprises a welded connection between the reinforcing braid and the structural element.

9. The medical catheter shaft of claim 8, wherein the selective removal of the polymeric jacket is controlled to create a desired size and shape of exposure of the reinforcing braid.

10. The medical catheter shaft of claim 8, wherein the structural element is a reinforcing assembly, wherein the reinforcing assembly is positioned within the proximal portion of the shaft, wherein the reinforcing assembly comprises:
a pair of diametrically opposed helical reinforcing sleeves extending through the proximal portion of the shaft; and
an anchor ring attached to a distal end of the reinforcing sleeves, the anchor ring comprising a ring structure.

11. The medical catheter shaft of claim 10, wherein the mechanical connection connects the reinforcing braid and the anchor ring.

12. The medical catheter shaft of claim 11, wherein the reinforcing sleeves establish reinforced passageways configured to provide radial and lateral support for one or more steering elements extending from the proximal region to the distal region.

13. The medical catheter shaft of claim 12, wherein the anchor ring includes passageways for the one or more steering elements extending from the proximal region to the distal region.

14. The medical catheter shaft of claim 8, wherein the structural element is an articulation structure positioned within the distal portion of the shaft, the articulation structure having a proximal end, wherein the articulation structure comprises:
a plurality of longitudinally-arranged articulation elements,
one or more steering wire lumens, wherein the steering wire lumens extend through the articulation structure, and
one or more reinforcing members extending through the articulation elements.

15. The medical catheter shaft of claim 13, wherein the mechanical connection connects the reinforcing braid to the proximal end of the articulation structure.

16. The medical catheter shaft of claim 8, wherein the structural element is a single helical cable that comprises a proximal end and distal end, wherein the single helical cable extends longitudinally through the proximal shaft, and wherein the single helical cable provides structural support to the proximal shaft, and wherein the mechanical connection connects the reinforcing braid to the distal end of the single helical cable.

17. A method of producing a shaft for a medical catheter, the method comprising:
providing a flexible tubular member comprising a reinforcing braid and a polymeric jacket disposed over the reinforcing braid, the flexible tubular member having a proximal portion, and a distal portion;
removing a portion of the polymeric jacket at an attachment location to expose a portion of the reinforcing braid;
positioning a structural element within the flexible tubular member adjacent to the exposed portion of the reinforcing braid; and
connecting the structural element to the exposed reinforcing braid at the attachment location to establish a secure bond between the structural element and the flexible tubular member.

18. The method of claim 17, wherein connecting the structural element comprises welding the structural element to the reinforcing braid.

19. The method of claim 18, wherein the structural element comprises a reinforcing assembly comprising a pair of diametrically opposed helical reinforcing sleeves and an anchor ring attached to a distal end of the reinforcing sleeves, and wherein positioning the structural element includes positioning the anchor ring adjacent to the attachment location, and wherein connecting the structural element includes welding the anchor ring to the reinforcing braid.

20. The method of claim 18, wherein the structural element comprises an articulation structure having a proximal end, and wherein positioning the structural element includes positioning the proximal end of the articulation structure adjacent to the attachment location, and wherein connecting the structural element includes welding the proximal end of the articulation structure to the reinforcing braid.
